(19) United States
(12) Patent Application Publication
(10) Pub. No.: US 2022/0232834 A1
(43) Pub. Date: Jul. 28, 2022

(54) PSEUDOMONAS CHLORORAPHIS SPECIES AND ITS USE IN THE CONTROL OF DISEASES CAUSED BY BACTERIA AND FUNGI

(71) Applicants: T3 BIOSCIENCE, LLC, MEQUON, WI (US); UWM RESEARCH FOUNDATION, INC., MILWAUKEE, WI (US)

(72) Inventors: Ching-Hong Yang, Mequon, WI (US); Xiangyang Liu, Shorewood, WI (US)

(21) Appl. No.: 17/587,166

(22) Filed: Jan. 28, 2022

Related U.S. Application Data
(60) Provisional application No. 63/142,691, filed on Jan. 28, 2021.

(51) Int. Cl. A01N 63/27; C12N 1/20

(52) U.S. Cl. CPC A01N 63/27; C12R 2001/38; C12N 1/20

(57) ABSTRACT

The present disclosure concerns methods of using Pseudomonas chlororaphis subsp. aurantiaca 1214-CHY4 (Accession No. PTA-126941), the cell broth and novel metabolites produced therefrom, that can inhibit the growth of a variety of microbial species for a variety of crops.

### CROSS-REFERENCE TO RELATED APPLICATIONS

This application claims priority to U.S. Patent Application Ser. No. 63/142,691, filed Jan. 28, 2021, entitled “PSEUDOMONAS CHLORORAPHIS SPECIES AND ITS USE IN THE CONTROL OF DISEASES CAUSED BY BACTERIA AND FUNGI,” the contents of which are herein incorporated by reference in its entirety.

### FIELD OF THE INVENTION

This present invention relates to a novel strain of the bacterial species P. chlororaphis subsp. aurantiaca 1214-CHY4 (1214-CHY4) and the application of the metabolites produced by this strain to protect against pathogenic bacteria and fungi of plants.

### BACKGROUND OF THE INVENTION

The control of many bacterial and fungal pathogens is challenging due to the resistance to the existing treatments. Clavibacter michiganensis subsp. michiganensis is a gram-positive bacterium that causes bacterial canker of solanaceous crops such as tomato, pepper, and eggplant (Sen et al., 2015). C. michiganensis subsp. michiganensis causes plant wilting, stunting, reduced yields, and eventually plant death. Yield losses vary with years, locations, cultivars, and phenological stages of host infection. For example, in Ontario, Canada, C. michiganensis subsp. michiganensis accounted for yield losses of up to 84% in commercial fields, whereas in artificially inoculated crops, yield losses varied from 46 to 93% (Poysa, 1993). The economic losses are also high: in Michigan, the bacterial canker has cost individual processing tomato growers as much as $300,000 in a single year (Hausbeck et al. 2000). As C. michiganensis subsp. michiganensis causes severe yield and economic losses; it is a quarantine microorganism in the European Union and many other countries (de Leon, et al. 2008). C. michiganensis subsp. michiganensis was first discovered in Michigan but is found globally wherever tomatoes are grown. It is the most important bacterial disease of tomatoes; yield losses can be severe. China, US (East of Mississippi), Brazil, and some parts of EU are significant areas for the disease. It was reported that the continuous use of streptomycin leads to emergence of resistant C. michiganensis subsp. michiganensis strains, challenging researchers to look for novel alternatives to control this plant pathogenic bacterium (Valenzuela, et al. 2019).

Apple scab caused by Venturia inaequalis is one of the most challenging diseases for growers to control. It requires the right timing of protectant sprays early in the season. Most growers need to spray 3 to 6 times, depending upon the weather conditions such as rainfall triggers release of ascospores for primary infection and the amount of inoculum in the orchard from the previous season. The disease is most prevalent in Eastern and upper Midwest growing areas in the US but is also found in Pacific Northwest and California. Two types of resistant genes have been reported for Venturia inaequalis (Schouten et al. 2014) Apple scab is a major problem in most apple-producing regions, including EU, Canada, and China.

Late blight caused by Phytophthora infestans is the most important globally disease of potatoes and also causes substantial losses in tomatoes. It can also infect other Solanaceous species and a range of ornamentals. The disease is found globally wherever potatoes are grown. It overwinters in crop residue and cull piles from the previous season. Late blight can be a devastating disease in potatoes, causing total loss of crop if uncontrolled. Under cool and wet conditions, the disease can cause complete plant/crop collapse in 7 to 10 days. As the disease tends to strike late in the growing season, it is especially damaging as the grower has already invested a significant amount of money and resources into the crop. Chemical fungicides are used extensively for late blight control with 6 to 10 fungicide treatments, including both older protectant active ingredients like mancozeb, copper, and chlorothalonil as well as newer actives, including phenylamides, carbamates, triazoles, strobilurines, and others. Resistance development can be rapid as multiple sprays are required during the growing season, and if growers rely on a single mode of action for repeated sprays, the microorganism quickly develops resistance. During the 1990s, several new strains of P. infestans were introduced into the US from Mexico, which have led to increased severities of the disease as well as higher degrees of resistance to fungicides due to sexual reproduction among the different strains (Inglis et al, 1996).

Botrytis cinerea attacks an extremely broad and diverse range of crops, with more than 200 crops documented as host species. Infection is favored by cool and wet weather in spring and summer. Some of the most important host species include vines, strawberries, tomatoes, cucurbits, beans, tree nuts, and tree fruits. A broad range of chemical fungicides is used against Botrytis as nearly all manufacturers target this important disease. Current microbial biofungicides approved for use against Botrytis include Trichoderma harzianum, Bacillus amyloliquefaciens, B. subtilis, Streptomyces griseoviridis, and Streptomyces lydicus. There are also plant extract-based biofungicides that actively against B. cinerea, such as Regalia from Marrone and Timorex from the Stockton Group (STK).

Bacterial spot, caused by Xanthomonas euvesicatoria, X. gardneri, X. perforans, and X. vesicatoria, is the most common disease seen in tomatoes (Potnis et al. 2015). Bacterial speck, caused by Pseudomonas syringae pv. tomato, is an increasing problem as well and it is a bigger problem when temperatures are cooler (Basim et al. 2004). In both cases, copper-based products or copper combined with mancozeb have been used as the primary control methods, but pest resistance to copper has been increasing. Some growers have resorted to using Actigard (acibenzolar-S-methyl) in rotation with copper, but this is a costly treatment for growers. Biological treatments like bacteriophages have not gained much market share in Florida due to cost and lack of performance.

Bacterial wilt, caused by Ralstonia solanacearum, is one of the major diseases of tomato and other solanaceous plants. The disease is known to occur in the wet tropics, subtropics, and some temperate regions of the world. It is one of the most damaging plant pathogens. This pathogen affects more than 300 plant species in 44 families globally, including a wide range of crop plants, ornamentals, and weeds (Li et al, 2006). Even though the biological control agents such as Pseudomonas and Bacillus have been tested for the bacterial wilt, they have no effects after 40 days of field planting (Li et al. 2006).

Walnut blight is caused by Xanthomonas arboricola pv. juglandis (Xaj). It is a major problem in California (Buchner et al. 2001). The application of copper for the treatment of walnut blight has resulted in the resistance of Xaj and side effects on the soil. Biological control of walnut blight pathogen by using kasugamycin is an alternative to chemical control. However, kasugamycin alone has more moderate efficacy and is at an elevated risk for Xaj resistance development. Current management of walnut blight in California includes the Cu-mancozeb, kasugamycin-mancozeb, kasugamycin-Cu rotation.

There is an imperative need for new multi-functional biopesticides derived from novel strains, cell broths and novel metabolites produced from such strains that can inhibit the growth of a variety of crop disease-causing pathogens.

### BRIEF SUMMARY OF THE INVENTION

In a first aspect, a method of growing bacteria to enhance production of protective metabolites is provided. The method includes a step of growing Pseudomonas chlororaphis subsp. aurantiaca 1214-CHY4 (1214-CHY4) (Accession No. PTA-126941) bacteria in liquid media in a vessel to produce a bacterial fermentate, wherein the vessel is shaken at a rate between about 150 and 250 RPM.

In a second aspect, an agricultural composition comprising the bacterial fermentate or the protective supernatant produced by any of the foregoing methods.

In a third aspect, a method of controlling bacterial crop diseases is provided. The method includes several steps. A first step includes producing an agricultural composition comprising the bacterial fermentate or the protective supernatant produced by any one of foregoing methods or any of the foregoing agriculture compositions. A second step includes applying said agricultural composition to crops to inhibit the growth of pathogenic microorganisms.

In a fourth aspect, a method of purifying at least one protective metabolite from Pseudomonas chlororaphis subsp. aurantiaca 1214-CHY4 (Accession No. PTA-126941) bacteria is provided. The method includes several steps. A first step includes producing a bacterial fermentate or protective supernatant or using their formulations. A second step includes extracting the bacterial fermentate or protective supernatant by ethyl acetate extraction. A third step includes producing an eluate containing at least one protective metabolite by eluting the bacterial fermentate or protective supernatant using 50% hexane and 50% ethyl acetate or by eluting the bacterial fermentate or protective supernatant using 25% hexane and 75% ethyl acetate (FIG. 2).

In a fifth aspect, a method of controlling bacterial crop diseases is provided. The method includes several steps. A first step includes producing an agricultural composition comprising at least one protective metabolite from Pseudomonas chlororaphis subsp. aurantiaca 1214-CHY4 (Accession No. PTA-126941) bacteria purified by one of the foregoing methods of the fourth aspect. A second step is applying said agricultural composition to crops to inhibit the growth of a pathogenic microorganism.

### BRIEF DESCRIPTION OF THE DRAWINGS

FIG. 1 illustrates an exemplary scheme for isolation and purification of Peaks 1-3.

FIG. 2 illustrates an exemplary scheme for isolation and purification of Peaks 4-5.

FIG. 3 illustrates bioactive metabolites produced by P. chlororaphis subsp aurantiaca 1214-CHY4.

### DETAILED DESCRIPTION

The present invention relates to a novel metabolite produced by Pseudomonas chlororaphis subsp. aurantiaca 1214-CHY4 (1214-CHY4) that exhibits antimicrobial activity against pathogenic microorganisms, including bacteria and fungi. The cell broth of this bacterial strain contains a novel natural products. These compounds, their method of production, and applications for inhibiting plant microbial pathogens are disclosed in greater detail herein.

### Definitions

When introducing elements of aspects of the disclosure or particular embodiments, the articles “a,” “an,” “the,” and “said” are intended to mean that there are one or more of the elements. The terms “comprising,” “including,” and “having” are intended to be inclusive and mean that there may be additional elements other than the listed elements. The term “or” means any one member of a particular list and also includes any combination of members of that list, unless otherwise specified.

As intended herein, the terms “substantially,” “approximately,” and “about” and similar terms are intended to have a broad meaning in harmony with the common and accepted usage in the art to which the subject matter of this disclosure pertains. It should be understood by those of skill in the art who review this disclosure that these terms are intended to allow a description of certain features described and claimed without restricting the scope of these features to precise numerical ranges provided. Accordingly, these terms should be interpreted as indicating that insubstantial or inconsequential modifications or alterations of the subject matter described and claimed are considered to be within the scope of the invention as recited in the appended claims.

“Biological control agents (or BCAs)” are a way of managing pests, such as pathogens, weeds, and insects, safely, sustainably, and cost-effectively. These agents are introduced into the environment to target a pest species, with the aim of reducing the pest's population or abundance in the environment.

“Biologicals” are preparations of living microorganisms (bacteria and yeasts) that produce colonies on the hosts. These microorganisms are applied mainly to slow the pathogen buildup during the epiphytic phase (Tianna et al. (2018)).

“Biorational” is a term applied to microbe-based biopesticides. These biopesticides are often made by fermenting microbial strains. Most of these products have both anti-bacterial and anti-fungal activity (Tianna et al. (2018)).

“Biopesticides” is defined by The US Environmental Protection Agency (EPA) to be pesticides derived from natural materials and categorizes them as either biochemical pesticides, containing substances that control pests by nontoxic mechanisms, microbial pesticides, consisting of microorganisms that typically produce bioactive natural products (BNPs), or plant-incorporated-protectants with activity produced by plants because of added genetic materials (Gwinn K. D. (2018)).

In the present invention, the term “strain of the invention” refers to the strain P. chlororaphis subsp. aurantiaca 1214-CHY4.

In a first aspect, a method of growing bacteria to enhance production of protective metabolites is provided. The method includes a step of growing Pseudomonas bacteria in liquid media in a vessel to produce a bacterial fermentate, wherein the vessel is shaken at a rate between about 150 and 250 RPM. In a first respect, the method further includes a step separating the liquid media from the bacteria after a period of time to produce a protective supernatant comprising the protective metabolites. In a second respect, the bacterial strain is Pseudomonas chlororaphis subsp. aurantiaca 1214-CHY4 (Accession No. PTA-126941). In a third respect, the growing temperature is between about 10 degrees C. and 35 degrees C. In a fourth respect, the liquid media for the production of cells is selected from glucose media, malt extract media, and yeast extract media. In a fifth respect, the growing temperature is between about 16 degrees C. and 28 degrees C. In a sixth respect, the bacteria are grown for a period of at 18 h to 2 days. In a seventh respect, the bacteria are grown for a period of at least 2 days.

In a second aspect, an agricultural composition comprising the bacterial fermentate or the protective supernatant produced by any of the foregoing methods. In a first respect, the formulation of the protective supernatant or its metabolites is selected from a solution (SL), a soluble powder (SP), a soluble granule (SG), a suspension concentrate (SC), a wettable powder (WP), a water dispersible granule (WG), a suspoemulsion (SE), a granule (GR) and an encapsulated formulation. In a second respect, the formulation of bacterial fermentate and cells is selected from a suspension concentrate (SC), a wettable powder (WP), and a water dispersible granule (WG).

In a third aspect a method of controlling bacterial crop diseases is provided. The method includes several steps. A first step includes producing an agricultural composition comprising the bacterial fermentate or the protective supernatant produced by any one of foregoing methods or any of the foregoing agriculture compositions. A second step includes applying said agricultural composition to crops to inhibit the growth of pathogenic microorganisms. In a first respect, the crop diseases are selected from the group consisting of grey mold, fire blight, citrus canker, soft rot, olive knot, tomato bacterial speck, bacterial canker or blast (stone and pome fruits), angular leaf spot of cucurbits, bacterial spot of peach, tomato bacterial spot, walnut blight, bacterial wilt, tomato canker, potato late blight, apple scab, bacterial leaf blight, and bacterial leaf streak. In a second respect, the pathogenic microorganism is selected from the group consisting of Mycosphaerella fijiensis, Botrytis cinerea, Erwinia amylovora (Ea) (especially the streptomycin-resistant E. amylovora strains), Xanthomonas axonopodis pv. citri (Xac), Pectobacterium parmentieri, Pectobacterium atrosepticum, Pectobacterium carotovorum subsp. brasiliensis, Pectobacterium carotovorum subsp. carotovorum, Dickeya dadantii, Pseudomonas savastanoi pv. savastanoi (Psv), Pseudomonas syringae pv. tomato, Pseudomonas syringae pv. syringae, Pseudomonas syringae pv. lachrymans, Xanthomonas campestris pv. pruni, Xanthomonas campestris pv. vesicatoria, Xanthomonas arboricola pv. juglandis, Ralstonia solanacearum, Clavibacter michiganensis subsp. michiganensis, Phytophthora infestans, Venturia inaequalis, Xanthomonas oryzae pv. oryzae, Xanthomonas oryzae pv. oryzicola and Xanthomonas citri pv. citri. In a third respect, the crop is selected from the group consisting of bananas, apples, pears, crabapples, citrus, potatoes, pumpkins, onions, rice, African violets, plant species of Cruciferae, Solanaceae, Cucurbitaceae including carrots, potatoes, tomatoes, eggplants, leafy greens, squashes and cucurbits, peppers and green peppers, olive, stone and pome fruit plants including olives, peaches, walnuts.

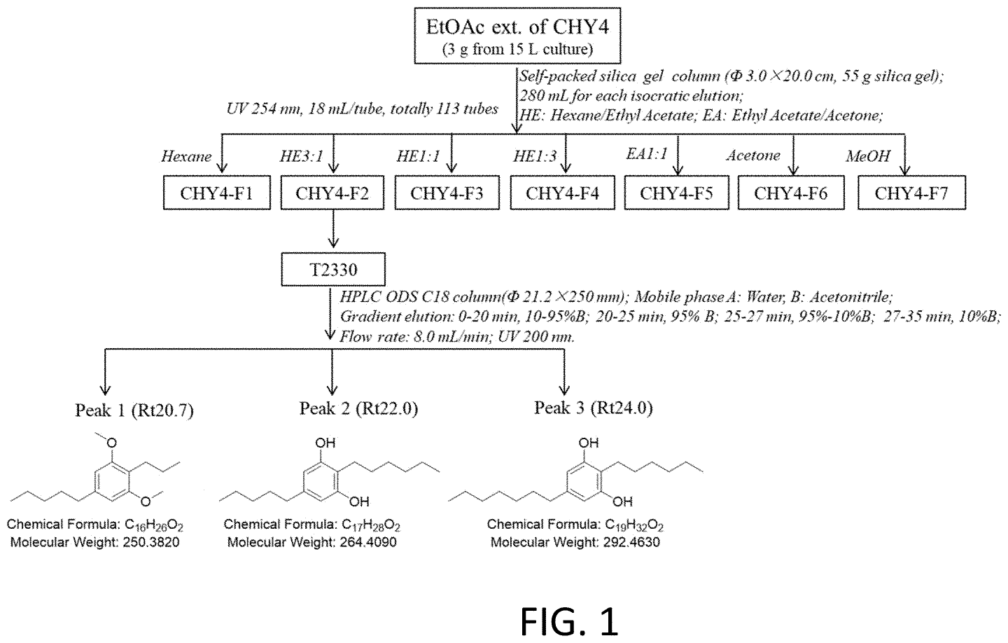

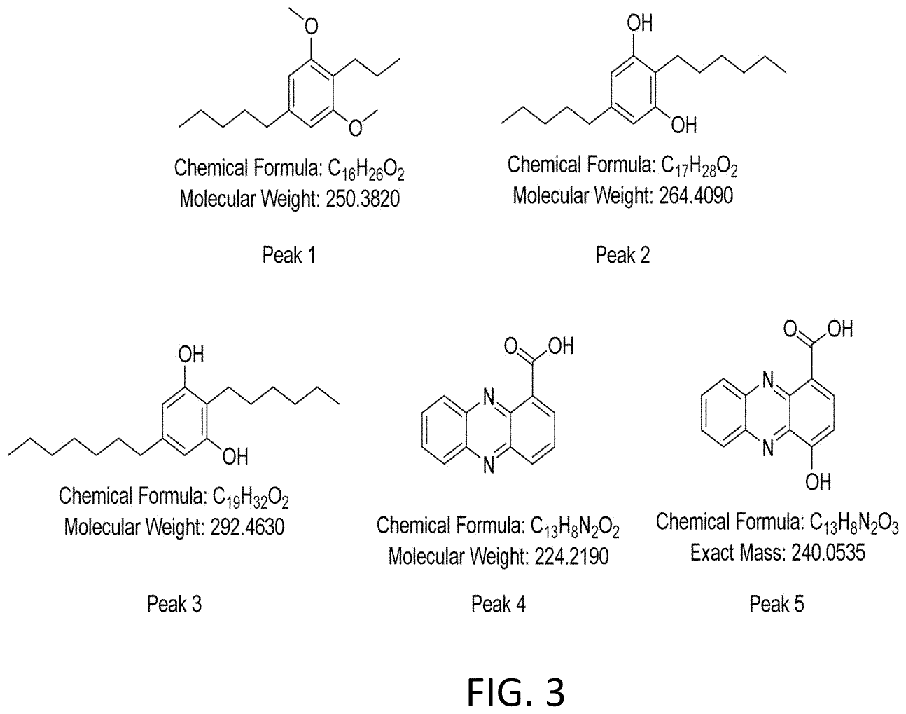

In a fourth aspect, a method of purifying at least one protective metabolite from Pseudomonas chlororaphis subsp. aurantiaca 1214-CHY4 (Accession No. PTA-126941) bacteria is provided. The method includes several steps. A first step includes producing a bacterial fermentate or protective supernatant or using their formulations. A second step includes extracting the bacterial fermentate or protective supernatant by ethyl acetate extraction. A third step includes apply the extract into a silica gel column (as in FIG. 1) and producing an eluate containing at least one protective metabolite by eluting the bacterial fermentate or protective supernatant using 50% hexane and 50% ethyl acetate or by eluting the bacterial fermentate or protective supernatant using 25% hexane and 75% ethyl acetate. In a first respect, the at least one protective metabolite is selected from peaks 1-5 or structural compounds derived therefrom, as depicted in FIG. 3.

In a fifth aspect, a method of controlling bacterial crop diseases is provided. The method includes several steps. A first step includes producing an agricultural composition comprising at least one protective metabolite from Pseudomonas chlororaphis subsp. aurantiaca 1214-CHY4 (Accession No. PTA-126941) bacteria purified by one of the foregoing methods of the fourth aspect. A second step is applying said agricultural composition to crops to inhibit the growth of a pathogenic microorganism. In a first respect, the crop disease is selected from the group consisting of grey mold, fire blight, citrus canker, soft rot, olive knot, tomato bacterial speck, bacterial canker or blast (stone and pome fruits), angular leaf spot of cucurbits, bacterial spot of peach, tomato bacterial spot, walnut blight, bacterial wilt, tomato canker, potato late blight, apple scab, bacterial leaf blight, and bacterial leaf streak. In a second respect, the pathogenic microorganism is selected from the group consisting of Mycosphaerella fijiensis, Botrytis cinerea, Envinia amylovora (Ea) (especially the streptomycin-resistant E. amylovora strains), Xanthomonas axonopodis pv. citri (Xac), Pectobacterium parmentieri, Pectobacterium atrosepticum, Pectobacterium carotovorum subsp. brasiliensis, Pectobacterium carotovorum subsp. carotovorum, Dickeya dadantii, Pseudomonas savastanoi pv. savastanoi (Psv), Pseudomonas syringae pv. tomato, Pseudomonas syringae pv. syringae, Pseudomonas syringae pv. lachrymans, Xanthomonas campestris pv. pruni, Xanthomonas campestris pv. vesicatoria, Xanthomonas arboricola pv. juglandis, Ralstonia solanacearum, Clavibacter michiganensis subsp. michiganensis, Phytophthora infestans, Venturia inaequalis, Xanthomonas oryzae pv. oryzae, Xanthomonas oryzae pv. oryzicola and Xanthomonas citri pv. citri. In a third respect, the crop is selected from the group consisting of bananas, apples, pears, crabapples, citrus, potatoes, tomatoes, eggplants, leafy greens, squashes and cucurbits, peppers and green peppers, olive, stone and pome fruit plants including olives, peaches, walnuts.

### Biological Deposit Information

The bacterial strain Pseudomonas chlororaphis subsp. aurantiaca 1214-CHY4 was submitted to the American Type Culture Collection (ATCC®), P.O. Box 1549, Manassas, Va. 20110 USA (“ATCC Patent Depository”) on Dec. 22, 2020. Following viability testing, the ATCC Patent Depository accorded this deposited bacterial strain the following Accession number, effective Dec. 22, 2020: Pseudomonas chlororaphis subsp. aureofaciens (now aurantiaca)1214-CHY4 (Accession No. PTA-126941).

One of the inventors, Dr. Ching-Hong Yang (residing at 10120 N. Sheridan Dr., Mequon, Wis. 53902, US), acting on behalf of Applicants, submitted the bacterial strain Pseudomonas chlororaphis subsp. aurantiaca 1214-CHY4 to the American Type Culture Collection (ATCC®), P.O. Box 1549, Manassas, Va. 20110 USA (“ATCC Patent Depository”) on Dec. 22, 2020, as evidenced by the Form PCT/RO/134, “Indications Relating to Deposited Microorganism,” pursuant to PCT Rule 13 bis (filed with this application). Following viability testing, the ATCC Patent Depository accorded this deposited bacterial strain the following Accession number, effective Dec. 22, 2020: Pseudomonas chlororaphis subsp. aureofaciens (now aurantiaca)1214-CHY4 (Accession No. PTA-126941). Dr. Yang grants permission to Applicants to include this biological deposit disclosure in the present application and gives his unreserved and irrevocable consent to it being made available to the public as of the date of filing.

### EXAMPLES

### Example 1. Isolation and Characterization of P. chlororaphis Subsp. Aurantiaca 1214-CHY4

1214-CHY4 is generally classified to the species Pseudomonas chlororaphis based on the search of the full length sequences of 16S rRNA, gyrB, rpoB and rpoD against the NCBI BLAST database. All four sequences of 1214-CHY4 show 99% identities to Pseudomonas chlororaphis strains (Table 1). 1214-CHY4 produces orange pigments that lead to the classification of subspecies either aureofaciens or aurantiaca and the strain cannot utilize 5-ketogluconate as the sole carbon source that further classifies the strain to subspecies aurantiaca (Peix et al, 2007). Therefore, the strain is classified as Pseudomonas chlororaphis subsp. aurantiaca 1214-CHY4.

TABLE 1
Search results of 1214-CHY4 16S sRNA/gyrB/rpoB/rpoD

| | | | Length of | |
|---|---|---|---|---|
| | | GenBank | full gene | |
| Gene | Top matched species | Sequence ID | sequence | Identities |
| 16S rRNA | Pseudomonas chlororaphis | CP045221.1 | 1532bp | 1531/1532 |
| | subsp. aurantiaca strain ARS 38 | | | (99%) |
| gyrB | Pseudomonas chlororaphis | KX696668.1 | 2418bp | 2398/2418 |
| | strain 48B8 | | | (99%) |
| rpoB | Pseudomonas chlororaphis | CP014867.1 | 4074bp | 4063/4074 |
| | isolate 189 | | | (99%) |
| rpoD | Pseudomonas chlororaphis | CP027712.1 | 1848bp | 1838/1848 |
| | subsp. chlororaphis strain DSM | | | (99%) |
| | 50083 | | | |

### Example 2. Culture, Crude Extract Preparation and Identification of Bioactive Compounds

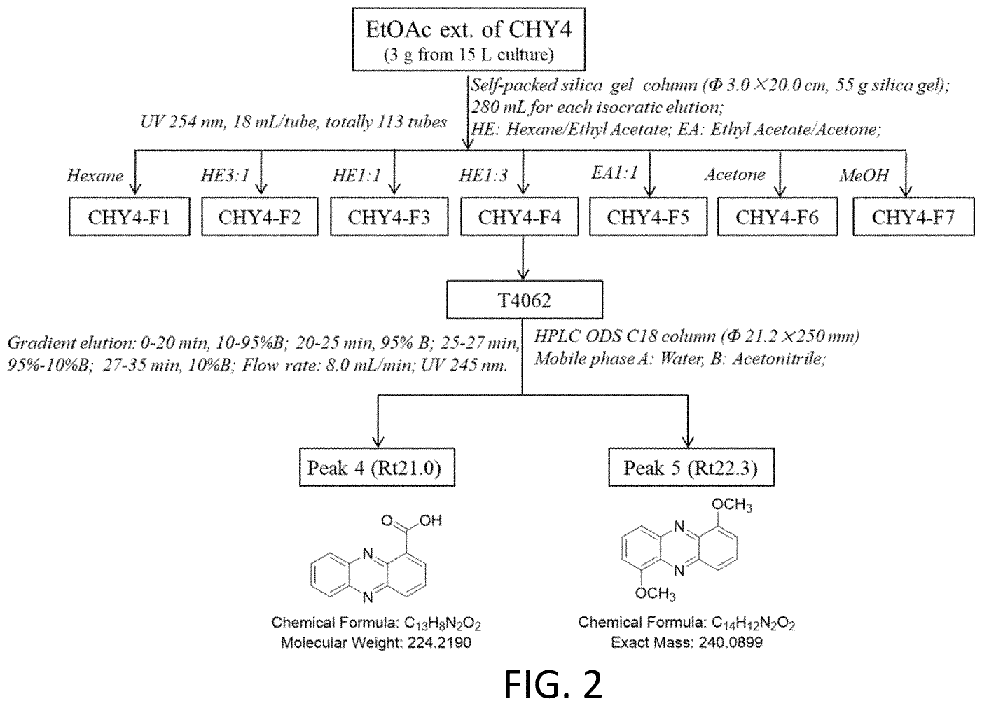

The strain P. chlororaphis subsp. aurantiaca 1214-CHY4 was streaked onto LB plate and cultivated in 28° C. incubator for one day. Several colonies were picked up and inoculated into YME medium in a fermenter for 3 days at 28° C. with an agitation speed at 200 rpm. The bacterial culture was extracted with an equal volume of ethyl acetate. The concentrated ethyl acetate extract solution was applied onto a Yamazen flash system (AI-580) equipped with a silica gel column (I.D. 3.0×20.0 cm, 55 g, 30μ, 60 Å) and separated by different concentrations of ethyl acetate/hexane (See FIG. 2).

The flash fractions were subjected to the antimicrobial test against Staphylococcus aureus, Venturia inaequalis, Phytophthora infestans, and Botrytis cinerea. The most active fractions are from Tube 21-30, Tube 37-62 (Table 2.1).

TABLE 2.1
Summary of the assay results for the 57 flash fractions

| | |
|---|---|
| Pathogen | Positive Flash Fractions |

| | | |
|---|---|---|
| Staphylococcus aureus | T22-T30 (T2230)^{a} | T40-T62 (T4062) |
| Venturia inaequalis | T21-T29 (T2129) | T37-T45 (T3745) |
| Phytophthora infestans | T23 | T37-T59 (T3759) |
| Botrytis cinerea | T27-T29 (T2729) | T39-T43 (T3943) |

^{a}‘T’ represents ‘Tube’. The samples from the odd number of flash fraction/Tubes were picked up to dry and dissolved in DMSO/MeOH, and then was used for the assay.

After HPLC purification of the active flash fractions, five bioactive compounds named Peak 1 (7.1 mg), Peak 2 (142 mg), Peak 3 (115.3 mg), Peak 4 (11.0 mg), and Peak 5 (3.0 mg) were obtained from 15 L of the 1214-CHY4 culture using preparative HPLC (FIG. 1, FIG. 2, FIG. 3). The structures of the 5 compounds were further determined by Liquid chromatographic-mass spectrometer (LCMS), High resolution-mass spectrometer (HR-MS), and Nuclear magnetic resonance (NMR) spectroscopy. LCMS and NMR data suggest that Peak 2 is 2-hexyl-5-pentylbenzene-1,3-diol which is also known as resorstatin. Resorstatin was reported as a free radical scavenging substance and has antimicrobial for various microbes (Table 2.2). The structure of Peak 3 is 2-hexyl-5-heptylbenzene-1,3-diol that was confirmed by X-ray crystallography analysis of the crystal structure in this disclosure. It has the same structure as compound 27 in the reported literature, but no crystal structure was available to confirm the structure of Peak 3. (Table 2.2). The backbones of Peak 2 and Peak 3 are related to the family of 2,5-dialkylresorcinols (DARs) including DB-2073 (Table 2.2). In 2021, a paper has reported that a Pseudomonas aurantiaca strain produced resorstatin (Compound 2 in the report) and Peak 3 (Compound 1 in the report) that both compounds have certain antimicrobial activity (Table 2.2). SciFinder search showed three journal articles (Table 2.2, Ref. [2,6,7]) mentioned the structure of Peak 3, but no other prior disclosures are reported for Peak 2 and Peak 3.

TABLE 2.2
Summary of the producers and activities of Peak 2, Peak 3 and DB2073

| Compounds | Producers | Activity (MIC values) | Ref¹ |
|---|---|---|---|
| Resorstatin | P. chlororaphis | See this disclosure for further information | Present |
| (2-hexyl-5- | subsp. aurantiaca | | disclosure |
| pentylbenzene- | 1214-CHY4 | | |
| 1,3-diol) | Pseudomonas sp. | Aspergillus fumigatus (50 μg/mL) | 1 |
| (Peak 2) | Ki19 | Candida albicans (50 μg/mL) | |
| | | Drechslera sorokiniana (50 μg/mL) | |
| | | Fusarium culmorum (50 μg/mL) | |
| | | Pseudomonas sevastanoi (50 μg/mL) | |
| | | Staphyllococcus aureus (7 μg/mL) | |
| | Pseudomonas sp. | Free radical scavenging substances | 5 |
| | DC165 | | |
| | Pseudomonas | Staphylococcus aureus | 6 |
| | aurantiaca | ATCC 12600 (2.1 μg/mL) | |
| | | Staphylococcus epidermidis | |
| | | ATCC 14990 (4.2 μg/mL) | |
| | | Enterococcus hirae | |
| | | ATCC 8043 (4.2 μg/mL) | |
| | | Streptococcus mutans | |
| | | ATCC 25175(4.2 μg/mL) | |
| | | Bacillus subtilis subsp. spizizenii | |
| | | ATCC 6633(4.2 μg/mL) | |
| 2-hexyl-5- | P. chlororaphis | See this disclosure for further information | Present |
| heptylbenzene- | subsp. aurantiaca | | disclosure |
| 1,3-diol | 1214-CHY4 | | |
| (Peak 3) | Azoarcus BH72 | Compound 27 of FIG. S3, antimicrobial | 2 |
| | | activity not provided | |
| | Pseudomonas | Staphylococcus aureus | 6 |
| | aurantiaca | ATCC 12600 (4.2 μg/mL) | |
| | | Staphylococcus epidermidis | |
| | | ATCC 14990 (4.2 μg/mL) | |
| | | Enterococcus hirae | |
| | | ATCC 8043 (8.4 μg/mL) | |
| | | Streptococcus mutans | |
| | | ATCC 25175(8.4 μg/mL) | |
| | | Bacillus subtilis subsp. spizizenii | |
| | | ATCC 6633(33.4 μg/mL) | |
| DB2073 | Pseudomonas sp. B- | Active against 22 bacteria and fungi, | 3-4 |
| | 9004 | including Gram-positive bacteria | |
| | | mycobacteria and Staphylococcus aureus | |
| | | (25 μg/mL) | |

¹References for this Table
1 Pohanka, A., Levenfors, J., & Broberg, A. (2006). Antimicrobial dialkylresorcinols from Pseudomonas sp. Ki19. Journal of Natural Products, 69(4), 654-657.
2 Fuchs, S. W., Bozhüyük, K. A. J., Kresovic, D., Grundmann, F., Dill, V., Brachmann, A. O., Waterfield, N. R., & Bode, H. B. (2013). Formation of 1,3-cyclohexanediones and resorcinols catalyzed by a widely occurring ketosynthase. Angewandte Chemie - International Edition, 52(15), 4108-4112.
3 Kanda, N., Ishizaki, N., Inoue, N., Oshima, M., Handa, A., & Kitahara, T. (1975). Db-2073, a new alkylresorcinol antibiotic. I. Taxonomy, isolation and characterization. The Journal of Antibiotics, 28(12), 935-942.
4 Kitahara, T., & Kanda, N. (1975). Db-2073, a new alkylresorcinol antibiotic. II. the chemical structure of Db-2073. The Journal of Antibiotics, 28(12), 943-946.
5 Kato, S., Shindo, K., Kawai, H., Matsuoka, M., & Mochizuki, J. (1993). Studies on free radical scavenging substances from microorganisms III. Isolation and structural elucidation of a novel free radical scavenger, resorstatin. Journal of Antibiotics, 46(6), 1024-1026.
6 Li, J., Shi, Y. & Clark, B. R. (2021). Semi-synthesis of antibacterial dialkylresorcinol derivatives. Journal of Antibiotics 74, 70-75.
[7] Budzikiewicz, H., Scholl, H., Neuenhaus, W., Pulverer, G. and Korth, H. (1980). “Dialkylresorcine aus Pseudomonas aureofaciens/Dialkyl Resorcinols from Pseudomonas aureofaciens” Zeitschrift für Naturforschung B, 35 (7), 909-910. (In German)

Peak 4 and Peak 5 are phenazine natural products. Peak 4 was analyzed by HR-MS and further confirmed by parallel analysis of the NMR spectra with the authentic compound phenazine-1-carboxylic acid (PCA). The PCA producer Pseudomonas aeruginosa M18 is an effective biocontrol agent that was isolated from the rhizosphere of sweet melon. The PCA yield of the genetically modified strain M18UMS/Phz reached approximately 4.7 g/L. PCA was commercially named shenqinmycin and a 1% shenqinmycin suspension was registered in China as an environmentally friendly fungicide (Product no. PD20110315) in 2011. This product was marketed in China to control rice and vegetable diseases caused by Rhizoctonia solani and Fusarium oxysporum (Table 2.3 and Table 2.4).

TABLE 2.3
Summary of the published producer and effects of Phenazine-
1-carboxylic acid on the pathogenic bacteria and fungi

| Producers | Active Against/Yield | Ref¹ |
|---|---|---|
| P. aeruginosa LV Strain | Botrytis cinerea | 1 |
| P. fluorescens LBUM636 | Phytophthora infestans | 2 |
| P. fluorescens LBUM223 | Phytophthora infestans | 3 |
| P. chlororaphis subsp. aureofaciens M71 | Seiridium cardinale | 4 |
| P. aeruginosa PA31x | Vibrio anguillarum C312 | 5 |
| P. fluorescens 2-79 | Gaeumannomyces graminis var. tritici. | 6 |
| P. fluorescens strain Psd | Fusarium oxysporum | 7 |
| P. aeruginosa MSH ΔlasR | ~250 mg/L | 8 |
| Pseudomonas sp. M18G | 1987 mg/L | 9 |
| Pseudomonas sp. M18UMS | 4.7 g/L | 10 |
| P. syringae pv. tomato (Pst) DC3000 | 13.4 μg/L | 11 |
| P. chlororaphis MCC2693 | Alternaria alternata > Phytophthora sp. > | 12 |
| | Fusarium solani > F. oxysporum | |

¹References for this Table
1 Simionato, A. S., Navarro, M. O. P., de Jesus, M. L. A., Barazetti, A. R., da Silva, C. S., Simões, G. C., Balbi-Peña, M. I., de Mello, J. C. P., Panagio, L. A., de Almeida, R. S. C., Andrade, G., & de Oliveira, A. G. (2017). The effect of phenazine-1-carboxylic acid on mycelial growth of Botrytis cinerea produced by Pseudomonas aeruginosa LV strain. Frontiers in Microbiology, 8(JUN), 1-9.
2 Morrison, C. K., Arseneault, T., Novinscak, A., & Filion, M. (2017). Phenazine-1-carboxylic acid production by Pseudomonas fluorescens LBUM636 alters Phytophthora infestans growth and late blight development. Phytopathology, 107(3), 273-279.
3 Roquigny, R., Novinscak, A., Arseneault, T., Joly, D. L., & Filion, M. (n.d.). Transcriptome alteration in Phytophthora infestans in response to phenazine-1-carboxylic acid production by Pseudomonas fluorescens strain LBUM223. BMC Genomics, 19, 474.
4 Raio, A., Reveglia, P., Puopolo, G., Cimmino, A., Danti, R., & Evidente, A. (2017). Involvement of phenazine-1-carboxylic acid in the interaction between Pseudomonas chlororaphis subsp. aureofaciens strain M71 and Seiridium cardinale in vivo. Microbiological Research, 199, 49-56.
5 Zhang, L., Tian, X., Kuang, S., Liu, G., Zhang, C., & Sun, C. (2017). Antagonistic activity and mode of action of phenazine-1-carboxylic acid, produced by marine bacterium Pseudomonas aeruginosa PA31x, against Vibrio anguillarum In vitro and in a zebrafish in vivo model. Frontiers in Microbiology, 8(FEB), 1-11.
6 Thomashow, L. S., Weller, D. M., Bonsall, R. F., & Pierson, L. S. (1990). Production of the antibiotic phenazine-1-carboxylic acid by fluorescent Pseudomonas species in the rhizosphere of wheat. Applied and Environmental Microbiology, 56(4), 908-912.
7 Upadhyay, A., & Srivastava, S. (2011). Phenazine-1-carboxylic acid is a more important contributor to biocontrol Fusarium oxysporum than pyrrolnitrin in Pseudomonas fluorescens strain Psd. Microbiological Research, 166(4), 323-335.
8 Sun, S., Zhou, L., Jin, K., Jiang, H., & He, Y. W. (2016). Quorum sensing systems differentially regulate the production of phenazine-1-carboxylic acid in the rhizobacterium Pseudomonas aeruginosa PA1201. Scientific Reports, 6(February), 16-18.
9 Li, Y., Jiang, H., Du, X., Huang, X., Zhang, X., Xu, Y., & Xu, Y. (2010). Enhancement of phenazine-1-carboxylic acid production using batch and fed-batch culture of gacA inactivated Pseudomonas sp. M18G. Bioresource Technology, 101(10), 3649-3656.
10 Du, X., Li, Y., Zhou, W., Zhou, Q., Liu, H., & Xu, Y. (2013). Phenazine-1-carboxylic acid production in a chromosomally non-scar triple-deleted mutant Pseudomonas aeruginosa using statistical experimental designs to optimize yield. Applied Microbiology and Biotechnology, 97(17), 7767-7778.
11 Li, W., Xu, Y. ping, Jean-Pierre, M., Xu, X., Qi, X. F., Gu, Y., & Cai, X. Z. (2016). Functional identification of phenazine biosynthesis genes in plant pathogenic bacteria Pseudomonas syringae pv. tomato and Xanthomonas oryzae pv. oryzae. Journal of Integrative Agriculture, 15(4), 812-821.
12 Jain, R., & Pandey, A. (2016). A phenazine-1-carboxylic acid producing polyextremophilic Pseudomonas chlororaphis (MCC2693) strain, isolated from mountain ecosystem, possesses biocontrol and plant growth promotion abilities. Microbiological Research, 190, 63-71.

TABLE 2.4
Summary of SciFinder search for Peak 5

| Name of the compound | Notes | Ref¹ |
|---|---|---|
| 4-hydroxy-5,10-dioxo-phenazine-1- | Intermediate of | 1 |
| carboxylic acid. | chemical synthesis | |
| 4-hydroxyphenazine-1-carboxylic acid | natural product from | 2 |
| | Pseudomonas | |
| 4-hydroxyphenazine-1-carboxylic acid | Intermediate of | 3 |
| | chemical synthesis | |
| 4-hydroxyphenazine-1-carboxylic acid | Intermediate of | 4 |
| | chemical synthesis | |

¹References for this Table
1 WO 2020035548. 2020, Redox-active compounds and uses thereof.
2 Roemer. (1981). Bacterial constituents. Part II. Phenazines from pseudomonads. Zeitschrift Für Naturforschung., 8.
3 Checchi, S. (1958). 5-Aminopyrazole derivatives. V. Synthesis of new tri- and tetraheterocyclic pyrazopyrimidopyridines and pyrazopyrimidopyridopyridazones. Gazzetta Chimica Italiana., 88.
4 Nakamura, S. (1958). Structure of griseolutein-B, a Streptomyces antibiotic. II. Decarboxylation and periodic acid oxidation. Chemical & Pharmaceutical Bulletin., 6.

### Example 3. Test for the Antimicrobial Activity of 1214-CRY4 and its Metabolites Against Plant Disease-Causing Bacteria and Fungi

We investigated the biological activity of the 1214-CHY4 metabolites against plant-associated pathogenic bacteria, including the Gram-positive bacterium Clavibacter michiganensis subsp. michiganensis (Cmm), which causes tomato canker. The biological activities of the crude extract of 1214-CHY4 were also tested against plant-associated gram-negative pathogenic bacteria, Erwinia amylovora (Ea), Ralstonia solanacearum (Rs), and Xanthomonas arboricola pv. juglandis (Xaj), etc.

The crude extract from 1214-CHY4 showed inhibition zones against a broad range of different types of phytopathogenic bacteria, including E. amylovora, R. solanacearum, C. michiganensis subsp. michiganensis, and X. arboricola pv. juglandis at 25 mg/mL and 5 mg/mL according to the plate diffusion assay results (Table 3.1).

The MIC values were studied for Peak 2, Peak 3 and Peak 4 against three fungal and four bacterial species. MIC values of 1214-CHY4 crude extract against Venturia inaequalis VI19-032, Phytophthora infestans 88069, and Botrytis cinerea CA17 are 31.3 μg/mL, 1.56 μg/mL, and 62.5 μg/mL, respectively (Table 3.2).

In terms of antibacterial activity, 1214-CHY4 crude extract, Peak 2, and Peak 3 are the most active metabolites against Clavibacter michiganensis subsp. michiganensis (Cmm), with a MIC value of 1.95 μg/mL, 1.56 μg/mL and 0.78 μg/mL, respectively. Additionally, Peak 4 showed the strongest activity against Ralstonia solanacearum K60 (tomato wilt), with a MIC value of 6.25 μg/mL (Table 3.2).

Due to the limited amount, Peak 1 was only tested against Venturia inaequalis, which shows 100% inhibition of Venturia inaequalis at 80 μg/mL (Table 3.3).

The overnight living bacterial culture of 1214-CHY4 at 1:500 dilution in PDA plate showed 100% antifungal activity against Venturia inaequalis, Phytophthora infestans Pi 1306, Botrytis cinerea CA17, Botrytis cinerea CA177, Botrytis cinerea CA31 (Table 3.3).

Peak 2 and Peak 3 have notable activity (1.56 μg/mL and 0.78 μg/mL respectively) against Cmm and have not been reported. Peak 1 is a novel structure with antifungal activity against Venturia inaequalis. Phenazine-1-carboxylic acid, is a potential antifungal compound, which has been reported to be effective against Botrytis cinerea and Fusarium oxysporum (Upadhyay and Srivastava, 2011); however, there is no report on the efficacy of this compound against Venturia inaequalis, the fungal pathogen causing apple scab. Our discovery demonstrates that the 1214-CHY4 metabolites are the biocontrol agents against tomato diseases and apple scab since they have nice activity against Cmm, R. solanacearum, and V. inaequalis (Table 3.2 and Table 3.3).

TABLE 3.1
Summary of the antimicrobial effect of 1214-CHY4
crude extract against phytopathogenic bacteria.

| | | Diameter of Growth Inhibition Zone (cm) | | | |
|---|---|---|---|---|---|
| | | CHY4 crude | CHY4 crude | | |
| | | extract | extract | Streptomycin | Vancomycin |
| Bacteria | Media | (25 mg/mL) | (5 mg/mL) | (200 μg/mL) | (200 μg/mL) |
| Erwinia amylovora 1189 (Fire | LB | 0.4 | 0.4 | 0.5 | NA |
| blight on apples/pears) | | | | | |
| Erwinia amylovora 110^{a} (Fire | LB | 0.5 | 0.3 | 0.5 | NA |
| blight on apples/pears) | | | | | |
| Erwinia amylovora DM1^{b} | LB | 0.5 | 0.4 | 0.0 | NA |
| (Fire blight on apples/pears) | | | | | |
| Erwinia amylovora CA11^{b} | LB | 0.5 | 0.4 | 0.0 | NA |
| (Fire blight on apples/pears) | | | | | |
| Erwinia amylovora 88^{c} (Fire | LB | 0.4 | 0.3 | 0.0 | NA |
| blight on apples/pears) | | | | | |
| Pectobacterium parmentieri | LB | 0.8 | 0.4 | 0.5 | NA |
| UPP163 936 (Causer of soft | | | | | |
| rot in multiple crops) | | | | | |
| Pectobacterium atrosepticum | LB | 0.3 | 0.4 | 0.0 | NA |
| 942 (Causer of soft rot in | | | | | |
| multiple crops) | | | | | |
| Pectobacterium carotovorum | LB | 0.7 | 0.3 | 0.0 | NA |
| subsp brasillensis 944 (Causer | | | | | |
| of soft rot in multiple crops) | | | | | |
| Pectobacterium carotovorum | LB | 0.5 | 0.3 | 0.0 | NA |
| subsp. carotovorum wpp14 | | | | | |
| 945 (Causer of soft rot in | | | | | |
| multiple crops) | | | | | |
| Dickeya dadantii 3937 | LB | 0.5 | 0.3 | 0.5 | NA |
| (Causer of soft rot in multiple | | | | | |
| crops) | | | | | |
| Pseudomonas savastanoi pv. | LB | 0.5 | 0.5 | 0.6 | NA |
| savastanoi (Olive knot) | | | | | |
| Pseudomonas syringae. pv | LB | 0.5 | 0.4 | 0.5 | NA |
| syringae 7046 (Bacterial | | | | | |
| canker or blast (stone and | | | | | |
| pome fruits)) | | | | | |
| Pseudomonas syringae. pv. | LB | 0.5 | 0.4 | 0.6 | NA |
| lachrymans 1188-1 (Angular | | | | | |
| leaf spot of cucurbits) | | | | | |
| Pseudomonas syringae. pv. | LB | 0.5 | 0.5 | 0.6 | NA |
| tomato PT30 (Tomato | | | | | |
| bacterial speck) | | | | | |
| Ralstonia solanacearum K60 | LB | 0.5 | 0.4 | 0.3 | NA |
| (Tomato wilt) | | | | | |
| Ralstonia solanacearum Pss4 | LB | 0.3 | 0.0 | 0.3 | NA |
| (Tomato wilt) | | | | | |
| Clavibacter michiganensis | LB | 1.1 | 1.0 | NA | 1.1 |
| subsp. michiganensis Cmm | | | | | |
| 0317 (Tomato canker) | | | | | |
| Clavibacter michiganensis | LB | 1.0 | 1.0 | NA | 1.2 |
| subsp. michiganensis Cmm | | | | | |
| 0690 (Tomato canker) | | | | | |
| Clavibacter michiganensis | LB | 1.3 | 1.1 | NA | 1.3 |
| subsp. michiganensis | | | | | |
| NCPPB382 (Tomato canker) | | | | | |
| Xanthomonas axonopodis pv. | NB | 0.4 | 0.4 | 2.2 | NA |
| citri N40-SO5 (Citrus canker) | | | | | |
| Xanthomonas axonopodis pv. | NB | 0.5 | 0.4 | 2.1 | NA |
| citri-Miami XC2002-00010 | | | | | |
| (Citrus canker) | | | | | |
| Xanthomonas campestris pv. | NB | 0.3 | 0.0 | 0.0 | NA |
| pruni (Bacterial spot of Peach) | | | | | |
| Xanthomonas campestris pv. | NB | 0.4 | 0.4 | 1.0 | NA |
| vesicatoria XV-16 (Tomato | | | | | |
| bacterial spot) | | | | | |
| Xanthomonas arboricola pv. | YGC | 0.3 | 0.3 | 0.0 | NA |
| juglandis 219 (Walnut blight) | | | | | |
| Xanthomonas arboricola pv. | YGC | 0.4 | 0.3 | 1.0 | NA |
| juglandis 417^{d} (Walnut blight) | | | | | |

^{a}Ea110 is the virulent strain used for the field trials in Michigan state;
^{b}Both CA11 and DM1 are streptomycin-resistant strains containing Tn5393 with the transposon on the acquired plasmid pEa34 and can grow in 100 μg/mL streptomycin containing media;
^{c}Ea88 is a spontaneous streptomycin-resistant strain with a mutation in the chromosomal rpsL gene and can grow in the media containing 2000 μg/mL streptomycin;
^{d}Copper resistant bacteria;
NA: Not available.

TABLE 3.2
Summary of MIC value study of 1214-CHY4 metabolites

| | MIC (μg/mL) | | | | | |
|---|---|---|---|---|---|---|
| Pathogen (Causing | Crude | | | | | |
| disease) | extract | Peak 2 | Peak 3 | Peak 4 | Vancomycin | Streptomycin |
| Venturia inaequalis | 31.3^{b} | >100^{b} | >100^{b} | 31.3^{b} | NA | NA |
| VI19-032 (Apple scab) | | | | | | |
| Phytophthora infestans | 1.56^{b} | >100^{b} | >100^{b} | 1.56^{b} | NA | NA |
| 88069 (Potato late | | | | | | |
| blight) | | | | | | |
| Botrytis cinerea CA17 | 62.5^{b} | >100^{b} | >100^{b} | 62.5^{b} | NA | NA |
| (Grey mold) | | | | | | |
| Ralstonia | >25000^{a} | >25000^{a} | >25000^{a} | 6.25^{b} | 100 | 25^{c} |
| solanacearum K60 | | | | | | |
| (Tomato wilt) | | | | | | |
| Pseudomonas syringae. | 180^{a} | 312^{a} | 156^{a} | 100^{b} | 100 | 3.13^{c} |
| pv. tomato PT30 | | | | | | |
| (Tomato bacterial | | | | | | |
| speck) | | | | | | |
| Xanthomonas | 180^{a} | 78^{a} | 78^{a} | 100^{b} | 50 | 50^{c} |
| euvesicatoria 75-3 | | | | | | |
| (Tomato spot) | | | | | | |
| Clavibacter | 1.95^{c} | 1.56^{c} | 0.78^{c} | 100^{b} | 0.39 | 12.5 |
| michiganensis subsp. | | | | | | |
| michiganensis 382 | | | | | | |
| (Tomato canker) | | | | | | |

^{a}The values were obtained by a modified disk diffusion method;
^{b}The values were obtained by agar dilution method;
^{c}The values were obtained by a modified broth dilution (microdilution) method;
NA: Not available.
Peak 4: The standard compound Phenazine-1-carboxylic acid (PCA) was used for the assay since Peak 4 was proved to be PCA.

TABLE 3.3
Summary of the antifungal study of 1214-CHY4 cells and peak 1.

| | Inhibitory % | |
|---|---|---|
| | 1214-CHY4 | Peak 1 |
| Fungi | (1:500 dilution)^{a} | (80 ug/mL) |
| Venturia inaequalis 19-032 | 100 | 100 |
| Phytophthora infestans Pi1306 | 100 | NA |
| Botrytis cinerea CA17 | 100 | NA |
| Botrytis cinerea CA177 | 100 | NA |
| Botrytis cinerea CA31 | 100 | NA |

^{a}1214-CHY4 cell culture grew at 28° C. overnight; then the cell culture was diluted to 1:500 in melted PDA media (40 μL cell broth in 20 mL PDA media in one petri dish plate). The results were checked on day 7 and day 14 for Venturia inaequalis and on day 3 for Phytophthora infestans and Botrytis cinerea.
NA: Not available.

### REFERENCES

Alzandi A. A., Naguib D. M. (2019) Pseudomonas fluorescens metabolites as biopriming agent for systemic resistance induction in tomato against Fusarium wilt, Rhizosphere 11:100168.
Anderson J. A., Staley J., Challender M., Heuton J. (2018) Safety of Pseudomonas chlororaphis as a gene source for genetically modified crops, Transgenic Research 27:103-113.
Arrebola E., Tienda S., Vida C., de Vicente A., Cazorla F. M. (2019) Fitness features involved in the biocontrol interaction of Pseudomonas chlororaphis with host plants: The case study of PcPCL1606, Frontiers in Microbiology 10:719.
Basim, H., Basim, E., Yilmaz, S., Dickstein, E. R., & Jones, J. B. (2004). An outbreak of bacterial speck caused by Pseudomonas syringae pv. tomato on tomato transplants grown in commercial seedling companies located in the western Mediterranean region of turkey. Plant Disease, 88(9), 1050.
Berdy B., Spoering A. L., Ling L. L., Epstein S. S. (2017) In situ cultivation of previously uncultivable microorganisms using the ichip, Nature Protocols 12:2232.
Buchner, R. P., Olson, W. H., & Adaskaveg, J. E. (2001). Walnut Blight (Xan thomonas Campestris pv. juglandis) Control Investigations In Northern California, USA. Acta Horticulturae, 544, 369-378.
David B. V., Chandrasehar G., Selvam P. N. (2018) Chapter 10-Pseudomonas fluorescens: A plant-growth-promoting rhizobacterium (PGPR) with potential role in biocontrol of pests of crops, in: R. Prasad, et al. (Eds.), Crop Improvement Through Microbial Biotechnology, Elsevier. pp. 221-243.
de Leon, L., Siverio, F., Lopez, M. M., and Rodriguez, A. (2008) Comparative efficiency of chemical compounds for in vitro and in vivo activity against Clavibacter michiganensis subsp. michiganensis, the causal agent of tomato bacterial canker. Crop Prot. 27:1277-1283.
Ganeshan G., Manoj Kumar A. (2005) Pseudomonas fluorescens, a potential bacterial antagonist to control plant diseases, Journal of Plant Interactions 1:123-134.
Gavrish E., Bollmann A., Epstein S., Lewis K. (2008) A trap for in situ cultivation of filamentous actinobacteria, Journal of Microbiological Methods 72:257-262.
Gerhardson B., Gustafsson A., Jerkeman T., Jingstrom B., Johnsson L., Hokeberg M. (1999) Composition and method for controlling plant diseases using Pseudomonas chlororaphis strain NCIMB 40616, Sweden.
Haas D., Défago G. (2005) Biological control of soil-borne pathogens by fluorescent pseudomonads, Nature Reviews Microbiology 3:307-319.
Hamamoto H., Urai M., Ishii K., Yasukawa J., Paudel A., Murai M., Kaji T., Kuranaga T., Hamase K., Katsu T., Su J., Adachi T., Uchida R., Tomoda H., Yamada M., Souma M., Kurihara H., Inoue M., Sekimizu K. (2015) Lysocin E is a new antibiotic that targets menaquinone in the bacterial membrane, Nature Chemical Biology 11:127-U68.
Hausbeck, M. K., Bell, J., Medina-Mora, C., Podolsky, R., and Fulbright, D. W. (2000) Effect of bactericides on population sizes and spread of C. michiganensis subsp. michiganensis on tomatoes in the greenhouse and on disease development and crop yield in the field. Phytopathology 90:38-44.
Hossain M. B., Piotrowski M., Lensing J., Gau A. E. (2009) Inhibition of conidial growth of Venturia inaequalis by the extracellular protein fraction from the antagonistic bacterium Pseudomonas fluorescens Bk3, Biological Control 48:133-139.
Inglis, D. A. Johnson, D. A., Fry, D. E. L., E., W. F., & P. B. Hamm. (1996). Relative resistances of potato clones in response to new and old populations of Phytophthora infestans. Plant Disease, 80, 575-578.
Kanda N., Ishizaki N., Inoue N., Oshima M., Handa A. (1975) DB-2073, a new alkylresorcinol antibiotic. I. Taxonomy, isolation and characterization, The Journal of Antibiotics 28:935-42.
Kato S., Shindo K., Kawai H., Matsuoka M., Mochizuki J. (1993) Studies on free radical scavenging substances from microorganisms. III. Isolation and structural elucidation of a novel free radical scavenger, resorstatin, The Journal of Antibiotics 46:1024-6.
Khokhani D., Zhang C. F., Li Y., Wang Q., Zeng Q., Yamazaki A., Hutchins W., Zhou S. S.,
Chen X., Yang C. H. (2013) Discovery of plant phenolic compounds that act as Type III secretion system inhibitors or inducers of the fire blight pathogen, Erwinia amylovora, Applied and Environmental Microbiology 79:5424-5436.
Kitahara T., Kanda N. (1975) DB-2073, a new alkylresorcinol antibiotic. II. The chemical structure of DB-2073, The Journal of Antibiotics 28:943-6.
Kumar S., Stecher G., Tamura K. (2016) MEGA7: Molecular evolutionary genetics analysis version 7.0 for bigger datasets, Molecular Biology and Evolution 33:1870-4.
Leisinger T., Margraff R. (1979) Secondary metabolites of the fluorescent pseudomonads, Microbiological Reviews 43:422-42.
Li, Y., Wang, W., Wei, H., Shen, G., Wang, R., Deng, J., Sun, X., & Shen, M. (2006). Microbial preparation & method for preventing and curing the bacterial wilt the plant and its use (Patent Application Publication No. US20060018883A1).
Peix, A., Valverde, A., Rivas, R., Igual, J. M., Ramirez-Bahena, M. H., Mateos, P. F., Santa-Regina, I., Rodriguez-Barrueco, C., Martinez-Molina, E., & Velázquez, E. (2007). Reclassification of Pseudomonas aurantiaca as a synonym of Pseudomonas chlororaphis and proposal of three subspecies, P. chlororaphis subsp. chlororaphis subsp. nov. P. chlororaphis subsp. aureofaciens subsp. nov., comb. nov. and P. chlororaphis subsp. au. International Journal of Systematic and Evolutionary Microbiology, 57, 1286-1290.
Pohanka A., Levenfors J., Broberg A. (2006) Antimicrobial dialkylresorcinols from Pseudomonas sp. Ki19, Journal of Natural Products 69:654-657.
Potnis, N., Timilsina, S., Strayer, A., Shantharaj, D., Barak, J. D., Paret, M. L., Vallad, G. E., & Jones, J. B. (2015). Bacterial spot of tomato and pepper: Diverse Xanthomonas species with a wide variety of virulence factors posing a worldwide challenge. Molecular Plant Pathology, 16(9), 907-920.
Poysa, V. (1993) Evaluation of tomato breeding lines resistant to bacterial canker. Can. J. Plant Pathol. 15:301-304.
Schouten, H. J., Brinkhuis, J., Burgh, A. Van Der, & Gessler, C. (2014). Cloning and functional characterization of the Rvi15 (Vr2) gene for apple scab resistance. Tree Genetics & Genomes, 10, 251-260.
Sen Y., van der Wolf J., Visser R. G. F., van Heusden S. (2015) Bacterial canker of tomato: current knowledge of detection, management, resistance, and interactions, Plant Disease 99:4-13.
Upadhyay A., Srivastava S. (2011) Phenazine-1-carboxylic acid is a more important contributor to biocontrol Fusarium oxysporum than pyrrolnitrin in Pseudomonas fluorescens strain Psd, Microbiological Research 166:323-335.
Valenzuela, M., Mendez, V., Montenegro, I., Besoain, X., & Seeger, M. (2019). Streptomycin resistance in Clavibacter michiganensis subsp. michiganensis strains from Chile is related to an rpsL gene mutation. Plant Pathology. 68: 426-433.

### INCORPORATION BY REFERENCE

All literature, publications, patents, patent applications, and related material cited here are incorporated by reference as if fully set forth herein.

1. A method of growing bacteria to enhance production of protective metabolites, comprising:
growing Pseudomonas bacteria in liquid media in a vessel to produce a bacterial fermentate, wherein the vessel is shaken at a rate between about 150 and 250 RPM.

2. The method of claim 1, further comprising separating the liquid media from the bacteria after a period of time to produce a protective supernatant comprising the protective metabolites.

3. The method of claim 1, wherein the bacterial strain is Pseudomonas chlororaphis subsp. aurantiaca 1214-CHY4 (Accession No. PTA-126941).

4. The method of claim 1, wherein the growing temperature is between about 10 degrees C. and 35 degrees C.

5. The method of claim 1, wherein the liquid media for the production of cells is selected from glucose media, malt extract media, and yeast extract media.

6. The method of claim 1, wherein the growing temperature is between about 16 degrees C. and 28 degrees C.

7. The method of claim 1, wherein the bacteria are grown for a period of at 18 h to 2 days.

8. The method of claim 1, wherein the bacteria are grown for a period of at least 2 days.

9. An agricultural composition comprising the bacterial fermentate or the protective supernatant produced by the method of claim 1.

10. The agricultural composition of claim 9, wherein the formulation of the protective supernatant or its metabolites is selected from a solution (SL), a soluble powder (SP), a soluble granule (SG), a suspension concentrate (SC), a wettable powder (WP), a water dispersible granule (WG), a suspoemulsion (SE), a granule (GR) and an encapsulated formulation.

11. The agricultural composition of claim 9, wherein the formulation of bacteria fermentate and cells is selected from a suspension concentrate (SC), a wettable powder (WP), and a water dispersible granule (WG).

12. A method of controlling bacterial crop diseases, comprising the steps of
i. producing an agricultural composition comprising the bacterial fermentate or the protective supernatant produced by claim 1 or the agriculture composition of claim 9; and
ii. applying said agricultural composition to crops to inhibit the growth of pathogenic microorganisms.

13. The method of 12, wherein the crop diseases are selected from the group consisting of grey mold, fire blight, citrus canker, soft rot, olive knot, tomato bacterial speck, bacterial canker or blast (stone and pome fruits), angular leaf spot of cucurbits, bacterial spot of peach, tomato bacterial spot, walnut blight, bacterial wilt, tomato canker, potato late blight, apple scab, bacterial leaf blight, and bacterial leaf streak.

14. The method of claim 12, wherein the pathogenic microorganism is selected from the group consisting of Mycosphaerella fijiensis, Botrytis cinerea, Envinia amylovora (Ea) (especially the streptomycin-resistant E. amylovora strains), Xanthomonas axonopodis pv. citri (Xac), Pectobacterium parmentieri, Pectobacterium atrosepticum, Pectobacterium carotovorum subsp. brasiliensis, Pectobacterium carotovorum subsp. carotovorum, Dickeya dadantii, Pseudomonas savastanoi pv. savastanoi (Psv), Pseudomonas syringae pv. tomato, Pseudomonas syringae pv. syringae, Pseudomonas syringae pv. lachrymans, Xanthomonas campestris pv. pruni, Xanthomonas campestris pv. vesicatoria, Xanthomonas arboricola pv. juglandis, Ralstonia solanacearum, Clavibacter michiganensis subsp. michiganensis, Phytophthora infestans, Venturia inaequalis, Xanthomonas oryzae pv. oryzae, Xanthomonas oryzae pv. oryzicola and Xanthomonas citri pv. citri.

15. The method according to claim 12, wherein the crop is selected from the group consisting of bananas, apples, pears, crabapples, citrus, potatoes, pumpkins, onions, rice, African violets, plant species of Cruciferae, Solanaceae, Cucurbitaceae including carrots, potatoes, tomatoes, eggplants, leafy greens, squashes and cucurbits, peppers and green peppers, olive, stone and pome fruit plants including olives, peaches, walnuts.

16. A method of purifying at least one protective metabolite from Pseudomonas chlororaphis subsp. aurantiaca 1214-CHY4 (Accession No. PTA-126941) bacteria, comprising:
i. producing a bacterial fermentate or protective supernatant by the method of any one of claims 1-8 or their formulations produced by claim 9;
ii. extracting the bacterial fermentate or protective supernatant by ethyl acetate extraction; and
iii. producing an eluate containing at least one protective metabolite by eluting the bacterial fermentate or protective supernatant from a self-packed silica gel column (Φ3.0×20.0 cm, 55 g silica gel) using 50% hexane and 50% ethyl acetate or using 25% hexane and 75% ethyl acetate.

17. The method of claim 16, wherein the at least one protective metabolite is selected from peaks 1-5 or structural compounds derived therefrom, as depicted below:

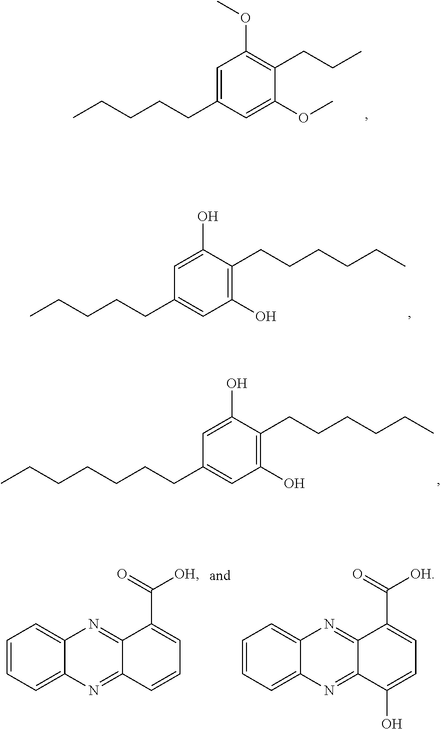

18. A method of controlling bacterial crop diseases, comprising:
i. producing an agricultural composition comprising at least one protective metabolite from Pseudomonas chlororaphis subsp. aurantiaca 1214-CHY4 (Accession No. PTA-126941) bacteria purified by the method of claim 15; and
ii. applying said agricultural composition to crops to inhibit the growth of a pathogenic microorganism.

19. The method of 18, wherein the crop disease is selected from the group consisting of grey mold, fire blight, citrus canker, soft rot, olive knot, tomato bacterial speck, bacterial canker or blast (stone and pome fruits), angular leaf spot of cucurbits, bacterial spot of peach, tomato bacterial spot, walnut blight, bacterial wilt, tomato canker, potato late blight, apple scab, bacterial leaf blight, and bacterial leaf streak.

20. The method according to claim 18, wherein the pathogenic microorganism is selected from the group consisting of Mycosphaerella fijiensis, Botrytis cinerea, Erwinia amylovora (Ea) (especially the streptomycin-resistant E. amylovora strains), Xanthomonas axonopodis pv. citri (Xac), Pectobacterium parmentieri, Pectobacterium atrosepticum, Pectobacterium carotovorum subsp. brasiliensis, Pectobacterium carotovorum subsp. carotovorum, Dickeya dadantii, Pseudomonas savastanoi pv. savastanoi (Psv), Pseudomonas syringae pv. tomato, Pseudomonas syringae pv. syringae, Pseudomonas syringae pv. lachrymans, Xanthomonas campestris pv. pruni, Xanthomonas campestris pv. vesicatoria, Xanthomonas arboricola pv. juglandis, Ralstonia solanacearum, Clavibacter michiganensis subsp. michiganensis, Phytophthora infestans, Venturia inaequalis, Xanthomonas oryzae pv. oryzae, Xanthomonas oryzae pv. oryzicola and Xanthomonas citri pv. citri.

21. The method of claim 18, wherein the crop is selected from the group consisting of bananas, apples, pears, crabapples, citrus, potatoes, tomatoes, eggplants, leafy greens, squashes and cucurbits, peppers and green peppers, olive, stone and pome fruit plants including olives, peaches, walnuts.
